(19) United States
(12) Patent Application Publication
(10) Pub. No.: US 2026/0191597 A1
(43) Pub. Date: Jul. 9, 2026

(54) METHOD AND SYSTEM FOR THREE-DIMENSIONAL SENSING BASED ON ONE-DIMENSIONAL SENSORS

(71) Applicant: EDDA TECHNOLOGY, INC., PRINCETON, NJ (US)

(72) Inventors: Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 19/010,547

(22) Filed: Jan. 6, 2025

(51) Int. Cl. A61B 34/20; G06T 7/246; G06T 7/73

(52) U.S. Cl. CPC A61B 34/20; G06T 7/246; G06T 7/73; A61B 2034/2055; A61B 2034/2065; A61B 2034/2072; A61B 2560/0223; G06T 2207/30004; G06T 2207/30204

(57) ABSTRACT

The present teaching relates to obtaining three-dimensional (3D) information in a contactless manner using a one-dimensional (1D) distance sensor. The 1D distance sensor is associated with an origin from where a laser beam emitted in an orientation, where the origin/orientation are calibrated with respect to a marker attached thereon and tracked. In a surgery, the calibrated 1D distance sensor is tracked by a camera via the marker attached thereon. When an emitted laser beam hits at a location on a patient, the 3D coordinate thereof in the tracker coordinate system is determined based on tracked marker, the origin/orientation of the 1D distance sensor, and a distance between the origin of the 1D distance sensor and the location.

### BACKGROUND

### 1. Technical Field

The present teaching generally relates to computers. More specifically, the present teaching relates to signal processing.

### 2. Technical Background

Knowing spatial information of a three-dimensional (3D) object is an essential need in many applications in various industries. Examples include unman vehicle or robot navigation. 3D location, shape, and volume of an object may be determined before controlling a robot to take an action, including obstacle avoidance, object grabbing, and manipulation of the object. Different commercial sensors have been developed to obtain 3D spatial information of an object. Such sensors are usually large in size and are usually intended for obtaining a dense 3D map of a target object. In some scenarios, what is needed may be 3D information for only a small set of locations of a target object. For instance, in a medical procedure, 3D information related to a patient on a surgical bed may be important to determine how to control a medical robot's movement. In this case, the 3D information on a few points on the patient may suffice. Some commercial hand-held sensors such as laser devices may allow to obtain some information. Most of such sensors provide only distance measures, e.g., a laser device may provide a distance between the sensor and the point that each laser beam hits a surface, without providing a 3D coordinate of the point when the laser beam encounters the object surface.

In addition, in most medical procedures, a location on a patient being operated on needs to be identified, e.g., a trocar point, where the cut is to be made. In some traditional solutions, such a location may be identified by introduce a physical touch using a probe with a tip which is calibrated with respect to a marker attached on the probe. As the patient is sanitized prior to the procedure, such a physical touch may introduce bacteria and, hence, not preferred.

Thus, there is a need for a solution that addresses these issues.

### SUMMARY

The teachings disclosed herein relate to methods, systems, and programming for information management. More particularly, the present teaching relates to methods, systems, and programming related to content summarization.

In one example, a method is disclosed for obtaining 3D information based on 1D sensor in a contactless manner. The 1D distance sensor is associated with an origin from where a laser beam emitted in an orientation, where the origin/orientation are calibrated with respect to a marker attached thereon and tracked. In a surgery, the calibrated 1D distance sensor is tracked by a camera via the marker attached thereon. When an emitted laser beam hits at a location on a patient, the 3D coordinate thereof in the tracker coordinate system is determined based on tracked marker, the origin/orientation of the 1D distance sensor, and a distance between the origin of the 1D distance sensor and the location.

In a different example, a system is disclosed for obtaining 3D information based on 1D sensor in a contactless manner. The 1D distance sensor is associated with an origin from where a laser beam emitted in an orientation. The system includes a calibration mechanism to calibrate the origin/orientation with respect to a marker attached thereon. The system also includes a application framework where the calibrated 1D distance sensor is used to determine the 3D coordinate of a location on a patient in a contactless manner. In application, the calibrated 1D distance sensor is tracked by a camera via the marker attached thereon. When an emitted laser beam hits at the location on a patient, the 3D coordinate thereof in the tracker coordinate system is determined based on tracked marker, the origin/orientation of the 1D distance sensor, and a distance between the origin of the 1D distance sensor and the location.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for obtaining 3D information based on 1D sensor in a contactless manner. The 1D distance sensor is associated with an origin from where a laser beam emitted in an orientation, where the origin/orientation are calibrated with respect to a marker attached thereon and tracked. In a surgery, the calibrated 1D distance sensor is tracked by a camera via the marker attached thereon. When an emitted laser beam hits at a location on a patient, the 3D coordinate thereof in the tracker coordinate system is determined based on tracked marker, the origin/orientation of the 1D distance sensor, and a distance between the origin of the 1D distance sensor and the location.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A shows an exemplary probe with a marker attached thereon;

FIG. 1B shows a tracking mechanism that tracks a probe via a marker attached thereon using a calibrated camera;

FIG. 1C illustrates an application in a surgery room to track a probe with a marker attached thereon using a calibrated camera;

FIG. 2A shows a one-dimensional (1D) laser-based distance measuring device;

FIG. 2B shows relevant parameters associated with a location of a 1D laser device;

FIG. 2C illustrates a 1D laser-based sensor having a marker attached thereon to be tracked, in accordance with an embodiment of the present teaching;

FIGS. 2D-2E illustrate different ways to attach a marker to a 1D distance sensor, in accordance with embodiments of the present teaching;

FIGS. 3A-3C depict a scheme to estimate an origin of a 1D distance sensor and an orientation of a laser beam emitted by the 1D distance sensor, in accordance with an embodiment of the present teaching;

FIG. 4A depicts an exemplary high level system diagram of a framework for estimating the origin/pose of a 1D distance sensor via tracking a marker attached thereto, in accordance with an embodiment of the present teaching;

FIG. 4B shows an application of determining a 3D coordinate of a laser beam intersection point in a contactless manner, in accordance with an embodiment of the present teaching;

FIG. 4C is a flowchart of an exemplary process of a framework for estimating the origin/pose of a 1D distance sensor via tracking a marker attached thereto, in accordance with an embodiment of the present teaching;

FIG. 5A illustrates a scheme of optimizing the estimation of the origin/pose of a 1D distance sensor, in accordance with an embodiment of the present teaching;

FIG. 5B shows a different scenario for optimizing the estimation of origin/pose of a 1D distance sensor, in accordance with an embodiment of the present teaching;

FIG. 6 is an illustrative diagram of an exemplary mobile device architecture that may be used to realize a specialized system implementing the present teaching in accordance with various embodiments; and

FIG. 7 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well-known methods, procedures, components, and/or systems have been described at a relatively high-level, without detail, in order to avoid unnecessarily obscuring aspects of the present teachings.

The present teaching discloses exemplary methods, systems, and implementations for acquiring 3D information via a 1D distance sensor. In some embodiments, the 1D distance sensor is a laser device that can be used to return a distance measure between the 1D distance sensor and a point on a surface that a laser beam emitted by the laser sensor hits. Given the distance reading from the 1D distance sensor, if the origin coordinate of the 1D distance sensor (where the laser is emitted) and the orientation of the laser beam are known, then the 3D information such as the coordinate of the point on the surface can be determined. In this manner, the 3D information may be acquired via the use of the 1D distance sensor.

The present teaching discloses an approach to estimating the 3D coordinate of the origin of a 1D distance sensor and the orientation of the laser beam emitted by the 1D distance sensor. A marker may be attached to the 1D distance sensor with a rigid spatial relation with respect to the origin of the 1D distance sensor. The mechanism as described herein is used to calibrate that rigid spatial relation between the marker and the origin so that so long as the position of the marker is known via, e.g., tracking, the position of the origin of the 1D distance sensor can also be determined. The orientation of the laser beam emitted by the 1D distance sensor can also be estimated and optimized using the same mechanism as described herein.

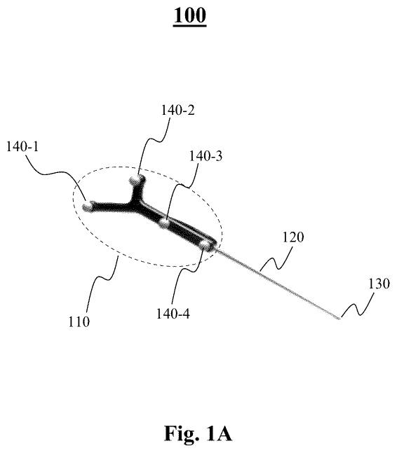

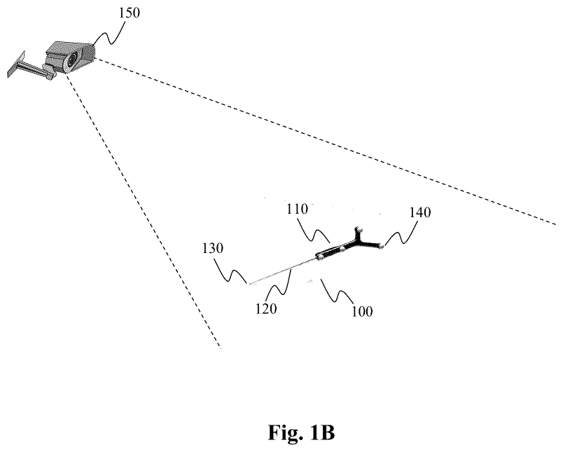

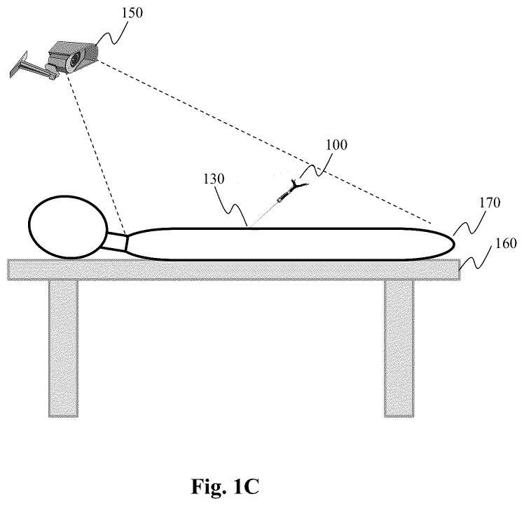

FIG. 1A shows an exemplary probe 100 with a marker attached thereon. In this illustration, a needle-like structure 120 has at one end a tip 130 and at the other end coupled with a marker 110, which includes one or more trackable units 140, e.g., 140-1 to 140-4, each of which may be tracked, and they may form a fixed spatial configuration. The spatial relationship between the tip 130 and the marker 110 is rigid and calibrated. That is, when the position of the marker 110 (or the positions of the trackable units on the marker) is known via, e.g., tracking, the position of the needle tip 130 can be accordingly determined based on the calibrated spatial relation. FIG. 1B shows a tracking mechanism with a camera 150 that tracks the probe 100 via its marker 110 attached thereon. In this set up, the camera 150 may be calibrated in a tracker coordinate system so that any point in the field of view has a calibrated coordinate in the tracker coordinate system. When the probe 100 is placed in the field of view of camera 150, the trackable units thereon 140 are captured in a camera image and corresponding locations for the trackable units in the image have corresponding 3D coordinate in the tracker coordinate system, which may then be based on to determine the 3D coordinate of the tip 130 based on known rigid spatial relations between the marker and the tip 130.

FIG. 1C illustrates an application of this tracking mechanism in a surgery room to track a probe. As shown, a patient 170 is on a surgical bed 160 for, e.g., an operation. In some scenarios, certain location on the patient 170 may be identified as significant such as a trocar point, at which a surgical instrument may be inserted to carry out the operation. In some situations, the 3D coordinate of the certain location needs to be determined, which may be done using the setting as shown in FIG. 1C, wherein a probe 100 is placed on the patient 170 at the specified certain location. As the marker 110 attached on the probe is captured by the camera 150, the 3D coordinate of the tip point 130 may be computed as discussed herein. The probe 100 with marker 110 as presented herein may also be used in other settings or applications. For example, it may be utilized to enable sensing 3D information using a 1D distance sensor.

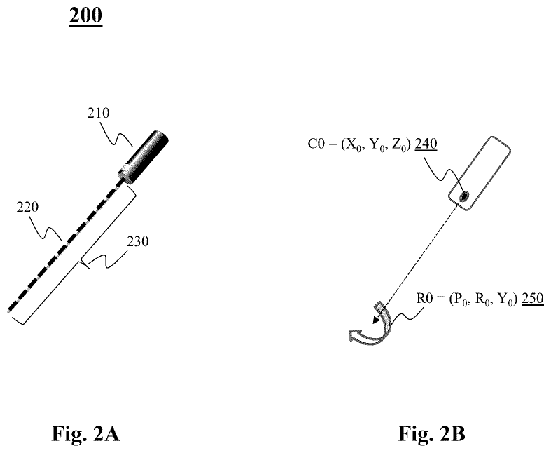

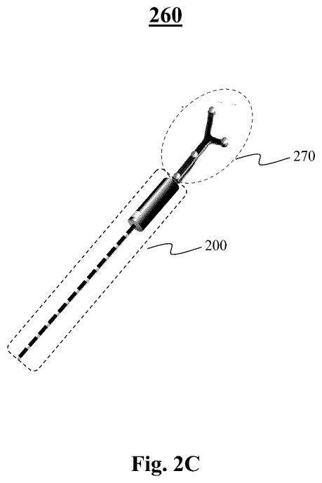

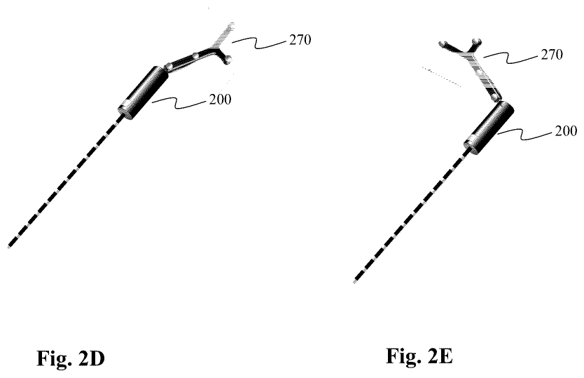

FIG. 2A shows a 1D laser-based distance sensor 200, with a body 210 that emits a 1D laser beam 220 (a line in space), analyzes the return signal, and outputs a reading represents a distance 230 between an origin of the device body 210 from where the laser beam is emitted and a surface (not shown) that the laser beam hits and is bounced. FIG. 2B shows some parameters associated with the 1D laser device 200 that are relevant to determine 3D information of the surface point based on the 1D laser beam 220. As shown, this includes a 3D coordinate Co=(Xₒ, Yₒ, Zₒ) of the origin 240 of the 1D distance sensor 210 and the rotation or orientation 250 of the laser beam 220, i.e., pitch P, roll R, and yaw Y represented as, e.g., Ro=(Pₒ, Rₒ, Yₒ). As discussed herein, the origin is where a laser beam is emitted by the 1D device, and the orientation of the laser beam characterizes the precise direction of the laser beam once it is emitted from the origin. Using such a 1D laser-based distance reading device, once the coordinate of the origin and the orientation of the laser beam are known, the distance reading from the origin and along the direction characterized by the orientation can be used to determine a 3D coordinate (3D information) of the point where the laser beam hits. That is, one can use a 1D distance sensor to sense 3D information so long as the origin coordinate and orientation of the beam are known.

FIG. 2C illustrates an exemplary 1D laser-based distance sensor 260 including a body 200 as shown in FIG. 2A with a marker 270 attached thereon to be tracked, in accordance with an embodiment of the present teaching. As discussed herein, the marker 270 is rigidly attached to the 1D distance sensor 210 with trackable units to allow their positions being tracked. In some embodiments, the trackable units may correspond to optical markers which allow an optical tracker such as a camera to detect. Other types of markers may also be used for the same purpose, as long as they can be detected in order to determine the pose of the rigid structure (1D laser distance sensor 210 in this case) to which the marker is attached. The marker 270 may be attached to the 1D distance sensor 210 in any arbitrary configurations and the rigid spatial relations between the marker 270 and the 1D distance sensor 210 may be calibrated accordingly. FIGS. 2D-2E illustrate different ways to attach marker 270 to 1D distance sensor 210, in accordance with embodiments of the present teaching. In each of the varying ways for the attachments, the spatial relation between the trackable units on the marker 270 and the origin of the 1D distance sensor 210 is different but may be calibrated in accordance with the present teaching as discussed herein.

In some embodiments, the marker 270 as attached to the 1D distance sensor 210 may be tracked by tracking each of the trackable units (e.g., 140-1 to 140-4) and then determine their respective positions. As discussed herein, as the trackable units are spatially configured with fixed spatial relationships with each other, their individual spatial relationships to the 1D distance sensor 210 may also be determined. In some embodiments, having multiple trackable units may be to ensure that at least one of them can be detected no matter of its pose. Each of the trackable units may have a rigid spatial relation with respect to the origin of the 1D distance sensor 210 and such rigid spatial relation may be estimated based on a process as discussed herein.

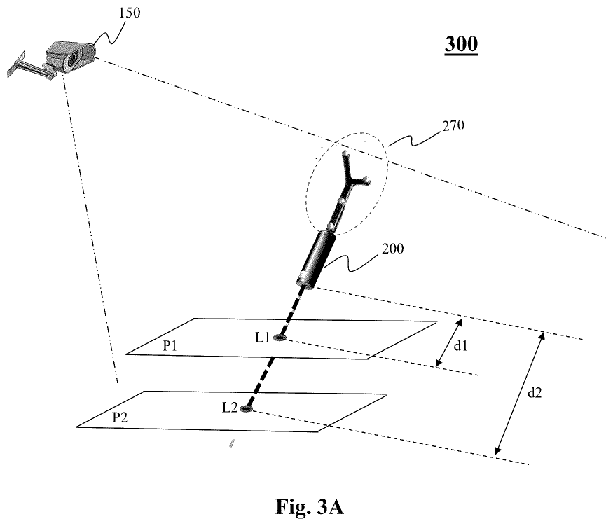

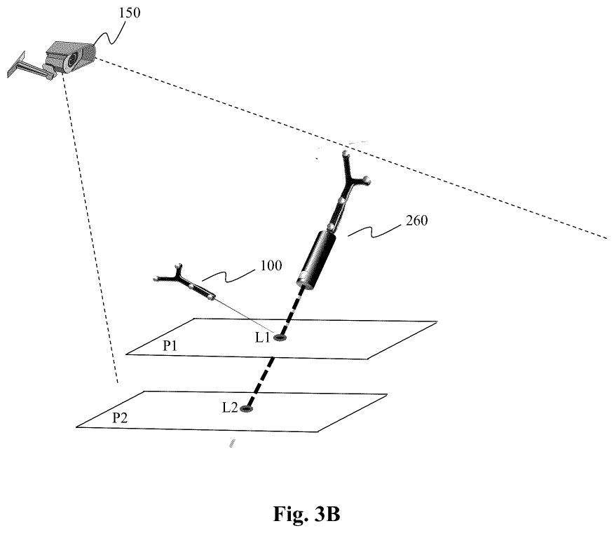

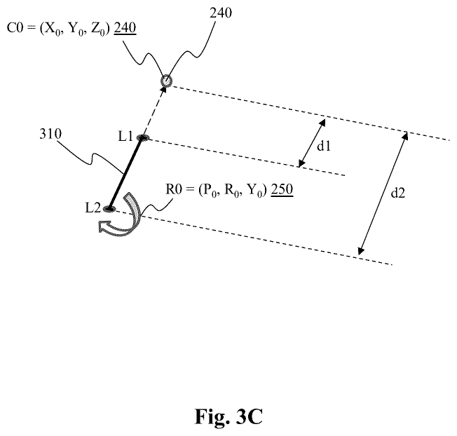

FIGS. 3A-3C depict a process to estimate the origin of a 1D distance sensor and the orientation of the laser beam emitted by the 1D distance sensor 210 from its origin, in accordance with an embodiment of the present teaching. FIG. 3A shows a set up in which a camera 150 is deployed with a field of view in a space 300, in which a 1D laser device 210 with attached marker is affixed in the space 300 in the field of view of the camera 150. The camera 150 may be calibrated with respect to the space 300. That is, in an image captured by the camera 150 within the field of view in 300, the 3D coordinate of each pixel in the image is known. The trackable units on marker 270 are visible to the camera 150 so that the coordinates of the trackable units on the marker 270 can be captured by the camera 150 and the coordinates of the visible trackable units may be determined in the tracker coordinate system. Through the tracking by the camera 150 in the tracker coordinate system, the position and orientation of the marker 270 can be tracked.

To determine the spatial relationship between the marker 270 and the origin of the 1D laser device 210, the origin and the coordinate thereof may be estimated first. In some embodiments, the following operational steps and relevant data processing may be performed to estimate the origin 240 of the 1D distance sensor 210 and the orientation of the laser beam emitted from the origin 250. The 1D distance sensor 210 may first be affixed in space 300. An object may be placed in front of the 1D distance sensor 210 in the path of the laser beam emitted by the 1D distance sensor. The object may be placed in a manner that will be hit directly by the laser beam. In some embodiments, the object corresponds to a planar object, as illustrated in FIG. 3A., In the following disclosure, an planar object is used, although any object may be used.

The surface of the planar object placed in front of the 1D distance sensor 210 may have a quality reflecting surface, such as white color or covered with white paper. The planar object is first placed at a location denoted by P1 in a way that when the affixed 1D distance sensor 210 emits a laser beam, it will hit the surface of the planar object to create an intersection point. For example, as shown in FIG. 3A, when the laser beam hits the planar object located at a point L1, the 1D distance sensor 210 reports a distance reading d1, representing the distance between the origin of 1D distance sensor 210 (which is unknown at this point) and point L1 on the surface of the planar object at P1. The coordinate of point L1 may be estimated in different ways. In some embodiments, when L1 is visible as a dot on the surface hit by the laser beam in space 300, the camera 150 may acquire an image with some pixel(s) therein corresponding the dot of the laser beam or L1. In this case, the pixel(s) representing L1 may be detected from the image. As camera 150 is calibrated, the 3D coordinate of L1 may be determined based on the 2D location of such pixel(s).

In some embodiments, the 3D coordinate of L1 may be determined using a probe such as probe 100 as discussed with reference to FIGS. 1A-1C. Probe 100 with attached marker 110 may be used to touch, via its tip 130, point L1 on the planar object at P1 as illustrated in FIG. 3B. As marker 110 attached on probe 100 is tracked by camera 150, the 3D coordinate of the tip 130 of the probe 100 can be determined based on the known spatial relationship between the probe marker 110 and the tip 130 of the probe. In this scenario, the 3D coordinate (X1, Y1, Z1) of the tip 130 of the probe 100 may be used to approximate the 3D coordinate of point L1.

After obtaining the estimated coordinate of point L1 in the tracker coordinate system, the planar object may be moved to a different location, e.g., at P2, which may either be farther away or closer to the 1D distance sensor 210 as compared with P1. It is noted that the planar object placed at P1 and P2 may or may not be parallel but they both need to be on the pathway of the laser beam emitted by the 1D distance sensor 210. FIG. 3A illustrates the scenario that P2 is farther away from the 1D distance sensor 210 than P1. In this scenario, the affixed 1D distance sensor 210 may be activated again to emit a laser beam to obtain a second distance reading d2, where d2>d1. It is noted that as the 1D distance sensor is affixed, the laser beams emitted to the planar object at P1 and P2 have the identical orientation so that they overlap in the shared portion. When the emitted laser beam hits the planar object at P2, it creates another intercepting point L2 on the planar object. The 3D coordinate of L2 (X2, Y2, Z2) may be similarly determined in either of the scenarios as discussed herein. In some embodiments, this process may repeat for N times, where N≥2, to obtain multiple points and corresponding coordinates.

Based on the 3D coordinates in the tracker coordinate system for both L1 and L2 (i.e., (X1,Y1, Z1) and (X2,Y2, Z2)), a line 310 in space 300 may be created, as shown in FIG. 3C. The origin 240 of the 1D distance sensor 210 as well as the orientation 250 of the laser beam emitted from the origin 240 may be estimated according to the present teaching. With respect to the origin 240, based on the 3D coordinate for point L1 and the distance reading d1, the origin of the 1D distance sensor 210 may be estimated by extending the line 310, from L1 and towards the 1D distance sensor 210, by distance d1 to arrive at estimated origin point 240. The origin may also be estimated based on L2 by extending line 310 towards 1D distance sensor 210 by a distance d2 to arrive at the estimated origin 240. The 3D coordinate for the estimated origin may then be computed based on, e.g., the equation for line 310 in the tracker space to obtain estimated Co=(Xₒ, Yₒ, Zₒ).

The equation for line 310 in the tracker coordinate system may be used to estimate the orientation 250 of the laser beam (as emitted by the 1D distance sensor 210 from the origin 240) with respect to a marker coordinate system of the marker 270. Given the coordinates of L1=(X1,Y1, Z1) and L2=and (X2,Y2, Z2) in the tracker coordinate system, the orientation of the laser beam in the tracker coordinate system may be computed as:

V_tracker=(L2_tracker-L1_tracker)/∥(L2_tracker-L1_tracker)∥

where the operator ∥.∥ represents the magnitude of a vector. As the position and orientation of the marker 270 are known in the tracker coordinate system (tracked by the camera 150), the orientation of the line 310 (representing the laser beam) may be computed with respect to the marker coordinate system. Assume that R_marker and T_marker denote the rotation matrix and translation vector of the marker coordinate system with respect to the tracker coordinate system, respectively. Then the orientation of the laser beam 310 in the marker coordinate system may be computed as:

$$V\_marker = {R\_marker}^{T} * V\_tracker$$

where R_marker^{T} is the transpose of R_marker.

Once the orientation 250 of the laser beam 310 in the marker coordinate system is obtained, the origin of the laser beam may also be computed. First, as discussed herein, the origin of the beam in the tracker coordinate system may be computed by tracing from, e.g., point L1 towards the 1D distance sensor 210 along line 310 (the laser beam) direction by a distance d1, expressed as:

$$O\_tracker = L1\_tracker = d 1 * V\_tracker$$

with the origin or O_tracker is so obtained, the origin in the marker coordinate system may be computed as:

$$O\_marker = {R\_marker}^{T} * O\_tracker - T\_marker$$

Although the above computations of the beam orientation and origin in the tracker/marker coordinate systems are illustrated with 2 points L1 and L2 on two planes, the scheme may be also applied when there are N points using, e.g., a least-square fitting to obtain more accurate estimates.

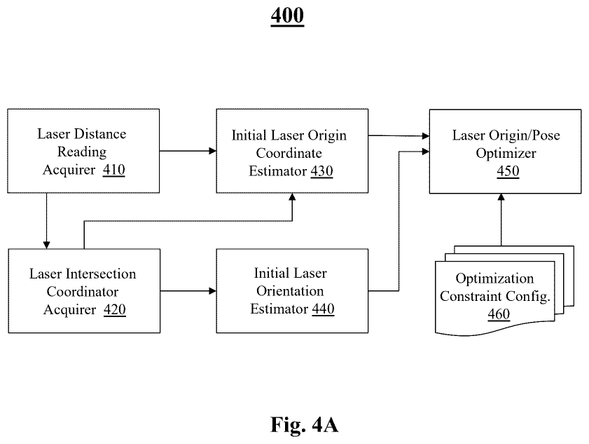

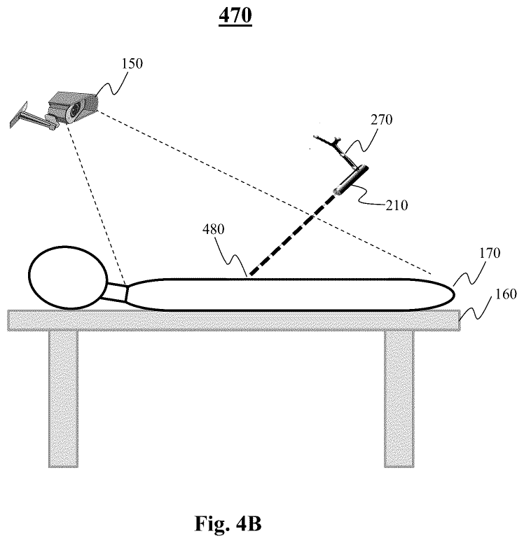

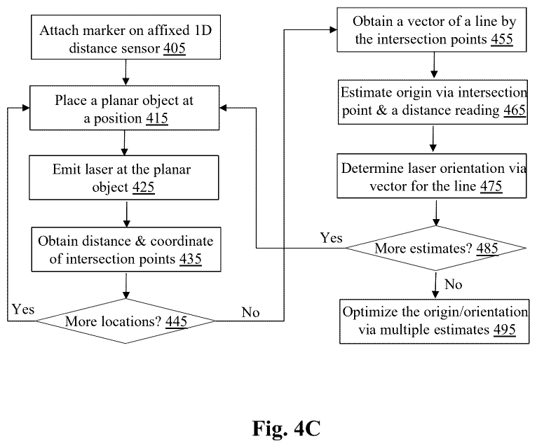

FIG. 4A depicts an exemplary high level system diagram of a framework 400 for estimating the origin of a 1D distance sensor and orientation of its laser beam, in accordance with an embodiment of the present teaching. The framework 400 comprises a laser distance reading acquirer 410, a laser intersection coordinate acquirer 420, an initial laser origin coordinate estimator 430, an initial laser orientation estimator 440, and a laser origin/pose optimizer 450. In this illustrated embodiment, the laser distance reading acquirer 410 may be connected to the affixed 1D distance sensor 210 to acquire a distance reading each time when the sensor is activated to produce a distance reading. The laser intersection coordinate acquirer 420 may be provided to estimate the coordinate of a point on a planar object hit by the laser beam emitted by the 1D distance sensor 210, e.g., coordinates (X1, Y1, Z1) and (X2, Y2, Z2) for L1 and L2 as illustrated in FIGS. 3A-3B in the tracker coordinate system. The initial laser origin coordinate estimator 430 may be provided to estimate the 3D coordinate of the origin of the 1D distance sensor 210 based on the 3D coordinates of the intersection points in the tracker space as discussed above. Similarly, the initial laser orientation estimator 440 may be provided for estimating the orientation of the laser beam emitted from the origin of the 1D distance sensor 210 based on the 3D coordinates of the intersection points in the tracker space as discussed herein.

Such obtained initial estimated origin and orientation as expressed in the coordinate system of the marker 270 may be used to determine the spatial relationships between the marker 270 and the origin of sensor 210 and between the marker 270 and the orientation of the sensor 210. Once such spatial relationships are determined, when the 1D distance sensor 210 with marker 270 attached thereon is deployed in an operation, by tracking the position of the marker 270, the position of the origin of the 1D distance sensor 210 as well as the orientation of the laser beam emitted from the origin may be determined based on the calibrated spatial relationships. When combined with the distance reading from the 1D distance sensor 210 in the operational setting, the 3D coordinate of the intersection point on the patient's skin can be determined in a contactless manner without touching the patient. This is shown in FIG. 4B, according to the present teaching. In this illustrated setting 470, a 1D distance sensor 210 with marker 270 attached thereon is deployed to obtain the 3D coordinate in the tracker coordinate system in the contactless manner, according to the present teaching. The marker on the 1D sensor is tracked and the 3D coordinate of a point 480 on a patient's skin intersected by a laser beam emitted from the origin of the sensor 210 can be computed based on tracked marker position, the calibrated spatial relationships, as well as the transformation matrices between the tracker and marker spaces.

To improve the precision of the estimated 3D coordinate of the intersection point on the patient's skin, it is important that the estimated origin of the 1D sensor 210 as well as the orientation of the laser beam emitted by 1D sensor 210 are accurate and precise. To ensure that, the laser origin/orientation optimizer 450 is provided for refining the estimates by optimization using a plurality of estimates subject to some constraint conditions. As discussed herein, the 3D coordinates of the intersection points are identified based on either the touching position of a probe 100 or the detected laser point position in an image. As these approaches may not be precise, the imprecision may be introduced in subsequent computations. Thus, an optimization method may be used. In some embodiments, this may be carried out by introducing multiple positions for origins and multiple orientations for the 1D distance sensor 210. For example, this may be achieved by emitting laser beams on a single plane from arbitrarily shifting the positions and changing the rotations of the tracked 1D distance sensor 210 to produce multiple 3D coordinates of estimated origin and multiple orientations of its laser beams at relevant origins. Such created intersection points are all on the single plane.

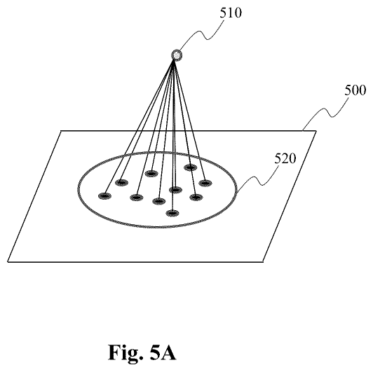

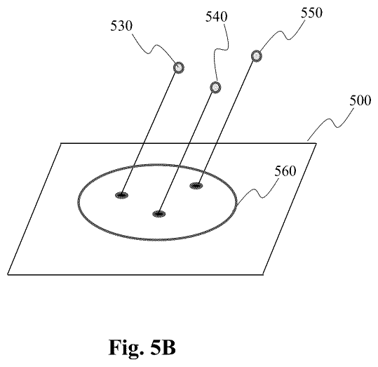

FIGS. 5A and 5B show exemplary schemes for creating intersection points on a single plane 500 to facilitate the optimization as described herein. FIG. 5A shows that a 1D distance sensor 210 positioned in a 3D location 510 (origin of the sensor 210) may be rotated arbitrarily around the fixed origin while emitting laser beams to produce a set of emitted laser beams with different corresponding orientations to produce a set 520 of intersection points, as seen in FIG. 5A. FIG. 5B illustrates a different way to produce multiple intersection points in 560 on the single plane 500. In this case, the 1D distance sensor 210 may be placed at different locations, e.g., 530, 540, . . . , 550, while emitting laser beams, each of which yields an intersection point on the single plane 500. In some embodiments, the two schemes (one with arbitrary rotations as in FIG. 5A and the other with arbitrary origin locations) may be combined to produce more intersection points to enhance the accuracy of the estimated origin and orientation associated with the 1D distance sensor 210.

With each of the intersection points, the origin and orientation of the sensor 210 may be estimated. Although estimates vary, the intersection points, however produced, are conditioned on that they satisfy the equation of the single plane 500. Assume that equation for the plane in the tracker coordinate system is, without loss of generality,

$$Nx * x + Ny * y + Nz * z + 1 = 0$$

where Nx, Ny, and Nz correspond to parameters associated with the plane. The equation may be re-written in a vector form as:

$$N\_plane * X + 1 = 0$$

where N_plane=(nx, ny, nz) and X=(x, y, z)^{T}. The operator * is a vector dot product. Suppose the orientation and the origin of the laser beam in the tracker space at one particular i-th time of measurement is n_tracker_i and o_tracker_i. The 3D coordinate of the laser intersection point in the tracker coordinate system may be expressed:

$$L\_i = o\_tracker \_i + D\_i * v\_tracker \_i$$

where D_i represents the unknown true distance (not the measured one). Because the intersecting point is on the plane, the following is obtained by plugging in the above equation to the plane equation:

$$N\_plane * ( o\_tracker \_i + D\_i * v\_tracker \_i ) + 1 = 0$$

Expand the above equation, the following is derived:

$$N\_plane * o\_tracker \_i + D\_i * N\_plane * v\_tracker \_i + 1 = 0$$

From the above expanded equation, the distance measure may be computed as:

$$D\_i = - N\_plate * o\_tracker / ( N\_plane * v\_tracker )$$

When there is a total of K measurements, and at the i-th time, the measured distance is d_i, i=1, 2, . . . ,K. From the distance measurements and the tracked positions of the markers, optimal laser beam orientation and origin may be obtained by minimizing the differences between the measured distances d_i and the predicted distances D_i. The predicted distance is a function of the laser beam position and orientation in the tracker coordinate system. In some embodiments, the optimization may be performed via, e.g., a least-square method using, e.g., non-linear optimization methods, such as gradient descent or Levenberg-Marquart algorithm. The initial estimates for the origin of the 1D distance sensor 210 and the orientation of the laser beam may be obtained as described herein. The optimization also involves optimizing the plane parameters (Nx, Ny, Nz), whose initial values may be obtained by fitting the laser point coordinates obtained by a plane based on the initial values of the beam orientation and origin as well as the tracked marker position.

FIG. 4C is a flowchart of an exemplary process of framework 400 for estimating the origin of the 1D distance sensor 210 and the orientation of the laser beams emitted from the origin, in accordance with an embodiment of the present teaching. As discussed herein, at 405, the 1D distance sensor is first affixed in space 300 with a marker attached thereon. A planar object is placed, at 415, at a position in space 300. The 1D distance sensor 210 is controlled to emit, at 425, a laser beam onto the planar object. At 435, the laser distance reading acquirer 410 obtains the distance reading from the 1D distance sensor 210 and the laser intersection coordinate acquirer 420 obtains the coordinate in the tracker coordinate system of the point on the planar object intersected by the laser beam. As discussed herein, this involves the placement of the planar object in front of the 1D distance sensor 210, reading of the distance measure returned by the sensor 210, and then using a probe 100 to touch, using its tip 130, the intersection point to acquire the 3D coordinate of the tip via tracking with the camera 150. It is then determined, at 445, whether the planar object is to be placed at a different position to produce another intersection point. If so, the process returns to 415 to move the planar object to a different position and steps 425-435 are repeated. As discussed herein, this may repeat for N times in order to obtain an adequate number of 3D coordinates for estimating the origin and orientation according to the present teaching.

When N intersection points are obtained based on the planar object being placed at different positions, the process proceeds to step 455 estimate the origin and orientation based on such intersection points. Based on the acquired 3D coordinates of the intersection points in the tracker coordinate system, a vector is obtained, at 455, for line 310 formed based on the intersection points. Using the vector so obtained, the initial laser origin coordinate estimator 430 estimates, at 465, the 3D coordinate of the origin in the tracker space according to the approach as discussed herein. Similarly, based on the vector of the line formed using intersection points, the initial laser orientation estimator 440 determines, at 475, the orientation of the laser beam of the 1D distance sensor. Once such initial estimates are made, it is determined, at 485, whether to produce additional set of estimates at arbitrary positions and rotations (as illustrated in FIGS. 5A-5B) to facilitate optimization. If so, the process returns to step 415 to create intersection points for estimating the origin and orientation based on a next set of arbitrary sensor position and rotation. If adequate number of sets of estimates are created for optimization, the process continues to step 495 to optimize the estimated origin of the 1D distance sensor 210 and the orientation of the laser beams emitted from the origin of the 1D sensor 210 to produce optimized estimates according to the disclosure provided herein.

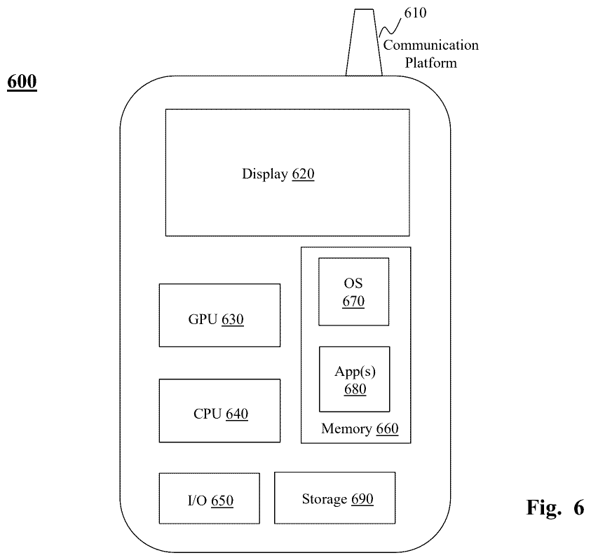

FIG. 6 is an illustrative diagram of an exemplary mobile device architecture that may be used to realize a specialized system implementing the present teaching in accordance with various embodiments. In this example, the user device on which the present teaching may be implemented corresponds to a mobile device 600, including, but not limited to, a smart phone, a tablet, a music player, a handled gaming console, a global positioning system (GPS) receiver, and a wearable computing device, or in any other form factor. Mobile device 600 may include one or more central processing units (“CPUs”) 640, one or more graphic processing units (“GPUs”) 630, a display 620, a memory 660, a communication platform 610, such as a wireless communication module, storage 690, and one or more input/output (I/O) devices 650. Any other suitable component, including but not limited to a system bus or a controller (not shown), may also be included in the mobile device 600. As shown in FIG. 6, a mobile operating system 670 (e.g., iOS, Android, Windows Phone, etc.), and one or more applications 680 may be loaded into memory 660 from storage 690 in order to be executed by the CPU 640. The applications 680 may include a user interface or any other suitable mobile apps for information analytics and management according to the present teaching on, at least partially, the mobile device 600. User interactions, if any, may be achieved via the I/O devices 650 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result, the drawings should be self-explanatory.

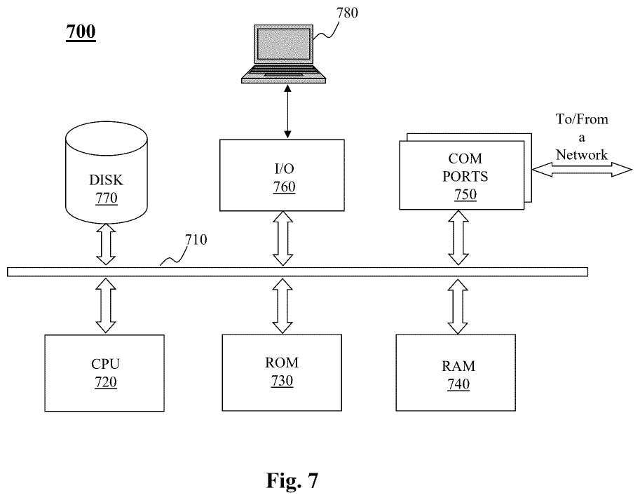

FIG. 7 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special-purpose computer. Both can be used to implement a specialized system for the present teaching. This computer 700 may be used to implement any component or aspect of the framework as disclosed herein. For example, the information analytical and management method and system as disclosed herein may be implemented on a computer such as computer 700, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 700, for example, includes COM ports 750 connected to and from a network connected thereto to facilitate data communications. Computer 700 also includes a central processing unit (CPU) 720, in the form of one or more processors, for executing program instructions. The exemplary computer platform includes an internal communication bus 710, program storage and data storage of different forms (e.g., disk 770, read only memory (ROM) 730, or random-access memory (RAM) 740), for various data files to be processed and/or communicated by computer 700, as well as possibly program instructions to be executed by CPU 720. Computer 700 also includes an I/O component 760, supporting input/output flows between the computer and other components therein such as user interface elements 780. Computer 700 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable the loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or a physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as the main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, a hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
deploying a camera in a tracking space, wherein the camera is for tracking in a tracker coordinate system;
affixing, within the tracking space, a one-dimensional (1D) distance sensor with a marker attached thereon, wherein the camera tracks the marker and the 1D distance sensor is used to measure a distance to an object by emitting laser from an origin in the 1D distance sensor according to an orientation;
estimating the origin and the orientation associated with the 1D distance sensor with respect to the marker;
deploying, in an operation on a patient, the 1D distance sensor with the marker attached thereon in the tracking space with the camera therein;
tracking, by the camera, the marker attached on the 1D distance sensor to obtain tracked marker information;
emitting, using the 1D distance sensor, a laser beam which hits the patient at a location;
obtaining a distance reading from the 1D distance sensor representing a distance from the origin of the 1D distance sensor to the location in a direction according to the orientation of the laser beam emitted from the origin; and
obtaining, in a contactless manner, a three-dimensional (3D) coordinate of the location on the patient in the tracker coordinate system based on the tracked marker information, the origin and orientation of the 1D distance sensor with respect to the marker, and the distance reading from the 1D distance sensor.

2. The method of claim 1, wherein the step of estimating the origin and the orientation with respect to the marker comprises:
tracking, using the camera, the marker attached on the 1D distance sensor to obtain marker information in the tracker space;
placing a first object at a first position in front of the affixed 1D distance sensor on the pathway of a laser beam emitted by the affixed 1D distance sensor;
emitting, using the 1D distance sensor, a first laser beam towards the first object at the first position to obtain a first distance;
detecting a first intersection point on the first object hit by the first laser beam;
placing a second object at a second position in front of the affixed 1D distance sensor on the pathway of a laser beam emitted by the affixed 1D distance sensor;
emitting, using the 1D distance sensor, a second laser beam towards the second object at the second position to obtain a second distance;
detecting a second intersection point on the second object hit by the second laser beam; and
determining the origin and the orientation based on the first and the second intersecting points and one of the first and the second distances.

3. The method of claim 2, wherein the step of determining the origin and the orientation comprises:
forming a line between the first and the second intersecting points;
identifying a point in the tracker space as an estimate for the origin by:
extending the line from the first intersection point towards the 1D distance sensor by the first distance to arrive at the point, or
extending the line from the second intersection point towards the 1D distance sensor by the second distance to arrive at the point; and
generating a vector corresponding to the line as the estimated orientation of the laser beam.

4. The method of claim 2, wherein
the marker includes a plurality of trackable units, each of which is tracked by the camera in the tracker space; and
the tracked marker information includes positions of the trackable units in the tracker coordinate system.

5. The method of claim 4, further comprising:
determining a first spatial relation between the estimated origin and the marker based on the tracked marker information; and
determining a second spatial relation between the estimated orientation and the marker based on the tracked marker information, wherein
the first spatial relation is used to determine a 3D coordinate of the origin with respect to the marker based on tracked marker information, and
the second spatial relation is used to determine the orientation of a laser beam emitted from the origin of the 1D distance sensor in the marker space based on tracked marker information.

6. The method of claim 5, wherein the step of obtaining the 3D coordinate of the location on the patient in the tracker coordinate system comprises:
determining a relative position of the origin of the 1D distance sensor with respect to the marker based on the tracked marker information and the first spatial relation;
determining a relative orientation of the 1D distance sensor based on the tracked marker information and the second spatial relation;
transforming the relative position of the origin with respect to the marker to the 3D coordinate of the origin in the tracker coordinate system; and
transforming the relative orientation with respect to the marker to the orientation of a laser beam emitted by the 1D distance sensor in the tracker coordinate system.

7. The method of claim 6, further comprising:
extending, the 3D coordinate of the origin in the tracker coordinate system along the orientation of a laser beam emitted by the 1D distance sensor by the distance as provided by the 1D distance sensor to reach a position in the tracker coordinate system; and
obtaining a coordinate in the tracker coordinate system corresponding to the reached position as the 3D coordinate of the location on the patient.

8. A machine-readable medium having information recorded thereon, wherein the information, when read by the machine, causes the machine to perform the following steps:
deploying a camera in a tracking space, wherein the camera is for tracking in a tracker coordinate system;
affixing, within the tracking space, a one-dimensional (1D) distance sensor with a marker attached thereon, wherein the camera tracks the marker and the 1D distance sensor is used to measure a distance to an object by emitting laser from an origin in the 1D distance sensor according to an orientation;
estimating the origin and the orientation associated with the 1D distance sensor with respect to the marker;
deploying, in an operation on a patient, the 1D distance sensor with the marker attached thereon in the tracking space with the camera therein;
tracking, by the camera, the marker attached on the 1D distance sensor to obtain tracked marker information;
emitting, using the 1D distance sensor, a laser beam which hits the patient at a location;
obtaining a distance reading from the 1D distance sensor representing a distance from the origin of the 1D distance sensor to the location in a direction according to the orientation of the laser beam emitted from the origin; and
obtaining, in a contactless manner, a three-dimensional (3D) coordinate of the location on the patient in the tracker coordinate system based on the tracked marker information, the origin and orientation of the 1D distance sensor with respect to the marker, and the distance reading from the 1D distance sensor.

9. The medium of claim 8, wherein the step of estimating the origin and the orientation with respect to the marker comprises:
tracking, using the camera, the marker attached on the 1D distance sensor to obtain marker information in the tracker space;
placing a first object at a first position in front of the affixed 1D distance sensor on the pathway of a laser beam emitted by the affixed 1D distance sensor;
emitting, using the 1D distance sensor, a first laser beam towards the first object at the first position to obtain a first distance;
detecting a first intersection point on the first object hit by the first laser beam;
placing a second object at a second position in front of the affixed 1D distance sensor on the pathway of a laser beam emitted by the affixed 1D distance sensor;
emitting, using the 1D distance sensor, a second laser beam towards the second object at the second position to obtain a second distance;
detecting a second intersection point on the second object hit by the second laser beam; and
determining the origin and the orientation based on the first and the second intersecting points and one of the first and the second distances.

10. The medium of claim 9, wherein the step of determining the origin and the orientation comprises:
forming a line between the first and the second intersecting points;
identifying a point in the tracker space as an estimate for the origin by:
extending the line from the first intersection point towards the 1D distance sensor by the first distance to arrive at the point, or
extending the line from the second intersection point towards the 1D distance sensor by the second distance to arrive at the point; and
generating a vector corresponding to the line as the estimated orientation of the laser beam.

11. The medium of claim 9, wherein
the marker includes a plurality of trackable units, each of which is tracked by the camera in the tracker space; and
the tracked marker information includes positions of the trackable units in the tracker coordinate system.

12. The medium of claim 11, wherein the information, when read by the machine, further causes the machine to perform the following steps:
determining a first spatial relation between the estimated origin and the marker based on the tracked marker information; and
determining a second spatial relation between the estimated orientation and the marker based on the tracked marker information, wherein
the first spatial relation is used to determine a 3D coordinate of the origin with respect to the marker based on tracked marker information, and
the second spatial relation is used to determine the orientation of a laser beam emitted from the origin of the 1D distance sensor in the marker space based on tracked marker information.

13. The medium of claim 12, wherein the step of obtaining the 3D coordinate of the location on the patient in the tracker coordinate system comprises:
determining a relative position of the origin of the 1D distance sensor with respect to the marker based on the tracked marker information and the first spatial relation;
determining a relative orientation of the 1D distance sensor based on the tracked marker information and the second spatial relation;
transforming the relative position of the origin with respect to the marker to the 3D coordinate of the origin in the tracker coordinate system; and
transforming the relative orientation with respect to the marker to the orientation of a laser beam emitted by the 1D distance sensor in the tracker coordinate system.

14. The medium of claim 13, wherein the information, when read by the machine, further causes the machine to perform the following steps:
extending, the 3D coordinate of the origin in the tracker coordinate system along the orientation of a laser beam emitted by the 1D distance sensor by the distance as provided by the 1D distance sensor to reach a position in the tracker coordinate system; and
obtaining a coordinate in the tracker coordinate system corresponding to the reached position as the 3D coordinate of the location on the patient.

15. A system, comprising:
a calibration mechanism including:
a camera, deployed in a tracking space, for tracking in a tracker coordinate system,
a one-dimensional (1D) distance sensor, affixed within the tracking space, with a marker attached thereon, wherein the camera tracks the marker and the 1D distance sensor is used to measure a distance to an object by emitting laser from an origin in the 1D distance sensor according to an orientation, wherein
the origin and the orientation associated with the 1D distance sensor are estimated and calibrated with respect to the marker;
a framework for determining, in a contactless manner, a three-dimensional (3D) coordinate of a location on a patient on a surgical table for an operation, including:
the 1D distance sensor with the marker attached thereon deployed in the tracking space, and
the camera for tracking the marker attached on the 1D distance sensor to obtain tracked marker information, wherein
the 1D distance sensor is used to emit a laser beam which hits the patient at the location to obtain a distance reading representing a distance from the origin of the 1D distance sensor to the location in a direction according to the orientation of the laser beam emitted from the origin, and
the 3D coordinate of the location in the tracker coordinate system is obtained based on the tracked marker information, the origin and orientation of the 1D distance sensor with respect to the marker, and the distance reading from the 1D distance sensor.

16. The system of claim 15, wherein the origin and the orientation with respect to the marker are estimated by:
tracking, using the camera, the marker attached on the 1D distance sensor to obtain marker information in the tracker space;
placing a first object at a first position in front of the affixed 1D distance sensor on the pathway of a laser beam emitted by the affixed 1D distance sensor;
emitting, using the 1D distance sensor, a first laser beam towards the first object at the first position to obtain a first distance;
detecting a first intersection point on the first object hit by the first laser beam;
placing a second object at a second position in front of the affixed 1D distance sensor on the pathway of a laser beam emitted by the affixed 1D distance sensor;
emitting, using the 1D distance sensor, a second laser beam towards the second object at the second position to obtain a second distance;
detecting a second intersection point on the second object hit by the second laser beam; and
determining the origin and the orientation based on the first and the second intersecting points and one of the first and the second distances.

17. The system of claim 16, wherein the step of determining the origin and the orientation comprises:
forming a line between the first and the second intersecting points;
identifying a point in the tracker space as an estimate for the origin by:
extending the line from the first intersection point towards the 1D distance sensor by the first distance to arrive at the point, or
extending the line from the second intersection point towards the 1D distance sensor by the second distance to arrive at the point; and
generating a vector corresponding to the line as the estimated orientation of the laser beam.

18. The system of claim 17, further comprising:
determining a first spatial relation between the estimated origin and the marker based on the tracked marker information; and
determining a second spatial relation between the estimated orientation and the marker based on the tracked marker information, wherein
the first spatial relation is used to determine a 3D coordinate of the origin with respect to the marker based on tracked marker information, and
the second spatial relation is used to determine the orientation of a laser beam emitted from the origin of the 1D distance sensor in the marker space based on tracked marker information.

19. The system of claim 18, wherein the 3D coordinate in the tracker coordinate system of the location on the patient is obtained by:
determining a relative position of the origin of the 1D distance sensor with respect to the marker based on the tracked marker information and the first spatial relation;
determining a relative orientation of the 1D distance sensor based on the tracked marker information and the second spatial relation;
transforming the relative position of the origin with respect to the marker to the 3D coordinate of the origin in the tracker coordinate system; and
transforming the relative orientation with respect to the marker to the orientation of a laser beam emitted by the 1D distance sensor in the tracker coordinate system.

20. The system of claim 19, further comprising:
identifying a position corresponding to the location in the tracker coordinate system by:
starting from the 3D coordinate of the origin in the tracker coordinate system,
extending, from the starting 3D coordinate, according to the orientation of the 1D distance sensor by the distance as read by the 1D distance sensor; and
obtaining a coordinate in the tracker coordinate system corresponding to the identified position as the 3D coordinate of the location on the patient.
